(19) United States
(12) Patent Application Publication
(10) Pub. No.: US 2019/0151269 A1
(43) Pub. Date: May 23, 2019

(54) MICROELEMENT VETERINARY PREPARATION

(71) Applicant: Andrei Anatolyevich Ryzhov, Tver (RU)

(72) Inventor: Andrei Anatolyevich Ryzhov, Tver (RU)

(21) Appl. No.: 15/818,781

(22) Filed: Nov. 21, 2017

(51) Int. Cl. A61K 31/198; A61K 33/26; A61K 33/06; A61K 33/32; A61K 33/34; A61K 33/30; A61K 33/24; A61K 33/04; A61K 33/00; A23K 20/20; A23K 20/24; A23K 20/142

(52) U.S. Cl. CPC A61K 31/198; A61K 33/26; A61K 33/06; A61K 33/32; A61K 33/34; A23K 20/142; A61K 33/24; A61K 33/04; A61K 33/00; A23K 20/30; A23K 20/24; A61K 33/30

(57) ABSTRACT

The invention refers to cattle breeding and veterinary medicine.

The preparation includes the following components, wt %:

2Na- or 2K-ethylenediamine-N, N1-disuccinic acid salt 20.0-50.0; Na- or K-amino-acid salt or amino-acid 3.0-15.0; iron (III) 0.3-3.0; magnesium (II); 0.3-3.0; manganese (II) 0.4-−2.5; copper (II) 0.05-0.25; zinc (II) 0.3-2.5; cobalt (II) 0.005-0.05; selenium (IV) 0.01-0.03; iodine (I) 0.03-0.08; the rest is water.

The preparation compensates deficiency of mineral nutrients, stimulates erythropoiesis, enhances nonspecific resistance of the body. The use of preparation enhances breeding capacity of animals, stimulates growth, development and productivity of live-stock and domestic animals.

### FIELD OF THE INVENTION

The invention is related to cattle breeding and veterinary medicine and can be used to prevent and treat iron deficiency anemia, iodine deficiency disorders, metabolic disorders, to enhance animal growth, to improve the reproductive function and immunity, and also to treat a number of specific diseases (oxidative stress, heavy metal intoxication, etc.).

### BACKGROUND OF THE INVENTION

The microelement veterinary preparation, Helavit (RU 2277800, 20.06.2006, Bulletin No. 17, A61K 31/295), is widely used. The preparation comprises a complex of iron, manganese, cobalt, copper, zinc, selenium and iodine with 2Na- or 2K-ethylenediamine-N,N¹-disuccinic acid salt and Na- or K-amino-acid salt selected from the group: glycine, alanine, valine, aspartic acid, glutamic acid, lysine, methionine, cystine, threonine, tryptophan, with the following ratio of ingredients (wt %): 2Na- or 2K-ethylenediamine-N,N1-disuccinic acid salt 15.0-35.0; Na- or K-amino-acid salt 2.0-10.0; iron (III) 0.6-3.0; manganese (II) 0.5-2.5; copper (II) 0.05-0.25; zinc (II) 0.3-2.5; cobalt (II) 0.005-0.05; selenium (IV) 0.01-0.03; iodine (I) 0.03-0.08; and the rest is water.

The preparation compensates deficiency of mineral nutrients, stimulates erythropoiesis, enhances nonspecific resistance of the body. The use of preparation enhances breeding capacity of animals, stimulates growth, development and productivity of live-stock and domestic animals.

However, as practice has shown, the animals' diet lacks such a vital element as magnesium. Magnesium ions are second most abundant elements, after potassium ions, they are involved in more than 600 enzymatic reactions, including protein synthesis and energy metabolism. Magnesium has an impact on regulation of biochemical processes in the body through magnesium-containing enzymes and free magnesium ions, being a cofactor of many enzymatic reactions (hydrolysis and phosphate group transfer, functioning of the Na+/K+-ATP pump, Ca2+-ATP pump, proton pump, participates in the exchange of electrolytes, ATP hydrolysis, reduces dissociation of oxidation and phosphorylation, regulates glycolysis and oxidation of fatty acids, participates in protein biosynthesis, transfer of genetic information, synthesis of cyclic AMP, participates in inflammatory reactions development), it forms a part of hemoglobin, numerous enzymes of nervous and glial tissues, and is involved in the processes of synthesis and degradation of neurotransmitters (noradrenaline, acetylcholine), as well as in the regulation of the neuromuscular activity of the body conducting tissues (cardiac muscle, skeletal muscles, unstriated muscles of visceras).

The studies conducted employing radioactive magnesium have shown that ruminants can sometimes metabolize only 10-20% of magnesium contained in herbs. Calves that for a long time were given unskimmed milk only can suffer from calcium metabolic imbalance in case of magnesium deficiency. Stamina of calves and the state of their health directly depend on the concentration of magnesium in the colostrum and on magnesium content in the diet of dry pregnant cows.

### SUMMARY OF THE INVENTION

The objective of the invention is to enhance efficacy of the preparation by adding magnesium missing in the animals' diets, to attain the balance of mineral nutrients in the composition.

To achieve this objective, the preparation based on the 2Na- or 2K-ethylenediamine-N,N1-disuccinic acid salt and Na- or K-amino-acid salt selected from the group: glycine, alanine, valine, aspartic acid, glutamic acid, lysine, methionine, cystine, threonine, tryptophan and salts of iron, manganese, copper, zinc, cobalt, selenium and iodine, was developed and magnesium was added in the form of an inorganic salt with the following ratio of ingredients (wt %):

2Na- or 2K-ethylenediamine-N,N1-disuccinic acid salt 20.0-50.0; Na- or K-amino-acid salt or amino-acid 3.0-15.0; iron (III) 0.3-3.0; magnesium (II); 0.3-3.0; manganese (II) 0.4-2.5; copper (II) 0.05-0.25; zinc (II) 0.3-2.5; cobalt (II) 0.005-0.05; selenium (IV) 0.01-0.03; iodine (I) 0.03-0.08; the rest is water.

The preparation contains inorganic salts of iron (III), magnesium (II), manganese (II), copper (II), zinc (II), cobalt (II), such as sulfates, chlorides, carbonates or oxides (in the form of hydrates or anhydrous salts). It contains selenious acid sodium or selenious acid as selenium salt (IV), and potassium iodide or sodium iodide or elemental iodine as iodide salt (I).

As an amino acid, the preparation contains an amino acid or Na- or K-amino acid salt of the general formula R—(NH2)COOH containing an amino group at the α-position to the carboxyl group. Monoaminocarboxylic acids (glycine, alanine, valine), monoaminodicarboxylic acids (aspartic, glutamic), diaminomonocarboxylic acid (lysine), thioamino acids (methionine, cystine), hydroxyamino acid (threonine), and heterocyclic amino acid (tryptophan) can be used as these compounds.

Thus, the proposed composition (Helamax preparation) is a biligand complex of an succinic acid derivative forming two 5-membered chelate rings with a metal ion and an amino acid (including an indispensable one) containing an amino group in the α-position to the carboxyl group and also forming a 5-membered chelate cycle with metal ions. The use of two ligands with different molecular weights, employed as transport molecules for the delivery of mineral nutrients through the cell membrane, enables to increase bioavailability of mineral nutrients by eliminating competition between mineral nutrients for absorption channels and protecting them from negative impact of the feed and gastrointestinal tract media.

The complex of mineral nutrients with the magnesium amino acid added to the system facilitates intake of additional nutritional components to enhance growth and development of animals. On the whole, this composition enables to normalize metabolism and strengthen erythrogenesis.

Adding less than 0.3 wt % of magnesium to the preparation does not significantly increase efficacy of the latter. If more than 3.0 wt. % is added, the preparation will become more expensive and the low molar mass of the element (2.3 times less than that of iron) will not allow to add sufficient amount of ligand according to the claim (total wt. % of substances will exceed 100%).

According to the invention, Helamax medication is prepared by ensuring interaction of an alkaline solution of 2Na- or 2K-ethylenediamine-N,N1-disuccinic acid salt and Na- or K-amino acid salt or an amino acid (pH 11-13) at a temperature of 60-70° C. with a calculated amount of salts of iron (III), magnesium (II), manganese (II), copper (II), cobalt (II), zinc (II), selenium (IV), and iodine (I). The resulting preparation is a dark brown liquid (pH 5-8). The resulting compound has an unlimited capacity to dissolve in water, but it does not dissolve in organic solvents. The preparation solution should be stored in a glass or plastic container. The composition obtained in the form of paste or powder by water evaporation is hygroscopic.

Ligand, ethylenediamine-succinic acid, is obtained through the reaction of amines nucleophilic addition through double bonds of unsaturated dicarboxylic acids. Ligand is synthesized by ensuring interaction of equivalent amounts of sodium or potassium salts of maleic acid and ethylenediamine (Patent of the Russian Federation No.

A microelement medication for animals is prepared by ensuring interaction of the ligand and glycine solution with the salts of mineral nutrients of iron (III), magnesium (II), copper (II), manganese (II), cobalt (II), zinc (II), selenium (IV) and iodine (I). Preliminarily (a total of 45.922%) 58.14 kg FeCl₃ (2.0%), 74.2 kg MgCl₂*6H₂O (0.89%), 43.8 kg MnSO₄*H₂O (1.4%), 37.5 kg of ZnCl₂ (1.8%), 7.86 kg of CuSO₄* 5H₂O (0.2%), 0.97 kg of CoCl₂*6H₂O (0.024%), 0.48 kg of NaSeO₃ (0.022%) and 0.68 kg KI (0.052%) are dissolved in softened water. The resulting solution of mineral nutrients salts is transferred to the chemical reactor containing an aqueous solution of 497.99 kg of ligand (43.8%) and 38.94 kg of glycine (3.89%), where everything is mixed up.

### DETAILED DESCRIPTION OF THE INVENTION

Acute and subchronic toxicity of the preparation was determined according to the “Guidelines for determination of toxic properties of medications used in cattle breeding and veterinary medicine” on white mice and rats. Based on the results of the experiments, the LD₅₀ value calculated according to the Kerber method exceeded 20,000 mg/kg of live weight, which, according to GOST 12.1.007.76, classifies the medication as Hazard Class 4 (slightly hazardous).

Efficacy of the medication can be illustrated by the following examples.

### EXAMPLE 1

Two groups of six-month puppies were formed from two litters of the same constitution and weight by the method of analog pairs. Throughout the experiment, experimental and control groups were fed the same food, the control group was additionally given Helavit (prototype), and the experimental group was given the proposed preparation (Helamax). The iron daily dosage of Helamax preparation and the prototype makes 1.0 mg per 1 kg of live weight. Puppies were weighed before the test and 40 days later.

Increased protein metabolism in the experimental group had a positive impact on the animals' growth rate (Table. 1).

TABLE 1
Growth dynamics of live weight of puppies, kg

| | Days | |
|---|---|---|
| Group (n = 10) | 0 | 40 |
| of experiment | 7.99 ± 0.15 | 13.22 ± 0.35 |
| of control | 8.13 ± 0.21 | 12.24 ± 0.41 |

The output parameters of the experimental group exceeded those of the control group by 8.01%.

The change in the content of humoral factors of the puppy body autarcesis is shown in Table. 2.

TABLE 2
The change in the content of humoral factors of the puppy body autarcesis

| | Days | | | | |
|---|---|---|---|---|---|
| Group | 0 | 10 | 20 | 40 | 65 |

Lysozyme activity, %

| | | | | | |
|---|---|---|---|---|---|
| of | 14.08 ± 0.44 | 16.89 ± 0.61 | 34.80 ± 0.89 | 28.19 ± 0.46 | 24.57 ± 0.83 |
| experiment | | | | | |
| of control | 14.01 ± 0.5 | 16.83 ± 0.54 | 30.57 ± 1.29 | 23.44 ± 0.21 | 20.71 ± 0.56 |

Bactericidal activity of blood serum, %

| | | | | | |
|---|---|---|---|---|---|
| of | 66.0 ± 2.28 | 93.2 ± 1.98 | 91.3 ± 2.15 | 91.6 ± 1.49 | 88.3 ± 2.25 |
| experiment | | | | | |
| of control | 64.8 ± 2.58 | 89.1 ± 1.22 | 82.9 ± 4.22 | 84.6 ± 1.15 | 81.2 ± 3.22 |

Table. 2 shows that the proposed composition has a more pronounced impact on the immune reactivity of the body. The maximum excess of serum bactericidal activity made 4.6% (the tenth day) and that of lysozyme activity hit 13.84% (the 20th day).

### EXAMPLE 2

Helamax efficacy was studied on 20 calves of black-motley breed at the age of 5-10 days, divided into 2 groups of 10 calves (according to the principle of analog pairs). Animals of both groups were fed in the same way and were kept under the same conditions. Calves of the control group were given Helavit (prototype) daily, 100 mg of iron per head, in addition to the main diet for 30 days, while the experimental group was given the proposed preparation in the same dosage. Clinical parameters of animals' blood are shown in Table 3.

TABLE 3
Indicators of dynamics of animal growth and clinical parameters of calves' blood

| | | | Average | | | | |
|---|---|---|---|---|---|---|---|
| | Average live | | daily | Hemoglobin, | | Erythrocytes, | |
| | weight, kg | | weight | g/l | | ×10¹²/l | |
| Group | Beginning | End | gain, g | Beginning | End | Beginning | End |
| of experiment | 35.2 | 55.8 | 686 + 48 | 83.5 + 4.2 | 132 + 9.5 | 4.9 + 0.2 | 6.1 + 0.8 |
| of control | 36.1 | 50.6 | 483 + 34 | 83.3 + 2.4 | 126 + 8.8 | 4.7 + 0.3 | 5.8 + 0.9 |

Table 3 confirms weight gain (42%) and more intensive stimulation of erythropoiesis in the animals of the experimental group who were given Helamax, so we can recommend the proposed preparation should be used in cattle breeding and veterinary medicine as an anti-anemic and growth-stimulating medication.

### EXAMPLE 3

The study was conducted on two groups of cows, 10 heads each group, which were fed in the same way, kept under the same conditions for 30 days and given same doses of iron.

TABLE 4
The content of magnesium of cows' blood serum after
administration of medications

| | | Group of animals | | |
|---|---|---|---|---|
| | | At the | | |
| | Measurement | beginning of | Helavit | Helamax |
| Indicator | units | experiment | (n = 10) | (n = 10) |
| Magnesium | mmol/1 | 0.73 ± 0.04 | 0.88 ± 0.3* | 1.34 ± 0.1* |

Note:
*accurate, compared to the values prior to administration of the medication.

After administering the medication, the magnesium concentration in the blood was normal in both groups of animals. In the group of animals who were given Helamax, the element concentration was 52.3% higher than in the group that was fed Helavit.

### EXAMPLE 4

Prevention of chronic intoxication with heavy metals was studied on a group of clinically healthy cows (n=15), aged 2-6 years, kept on a private farm. The medication was given with feed at a dose of 12 mg per 10 kg of live weight for 30 days.

TABLE 5
Impact of the medication on the content of heavy metals in the blood

| | | After | After |
|---|---|---|---|
| | Before | administration of | administration |
| Indicator | administration | Helavit | of Helamax |
| Cadmium, μg/ml | 4.32 ± 1.15 | 1.21 ± 0.91* | 1.01 ± 0.21* |
| Lead, μg/ml | 34.24 ± 4.92 | 18.35 ± 1.36* | 15.22 ± 1.11* |

Conclusion: The empirical data indisputably confirms high efficacy of the proposed medication (Helamax) for prevention and treatment of chronic intoxication with heavy metals.

1. A microelement veterinary preparation containing 2Na- or 2K-ethylenediamine-N,N1-disuccinic acid salt and Na- or K-amino-acid salt or amino-acid selected from the group: glycine, alanine, valine, aspartic acid, glutamic acid, lysine, methionine, cystine, threonine, tryptophan and salts of iron, manganese, copper, zinc, cobalt, selenium and iodine, which characteristic feature is magnesium being added, with the following ratio of ingredients (wt %):

| | |
|---|---|
| 2Na- or 2K-ethylenediamine-N, | 20.0-50.0 |
| N¹-disuccinic acid salt | |
| Na- or K-amino-acid salt or amino-acid | 3.0-15.0 |
| Iron (III) | 0.3-3.0 |
| Magnesium (II) | 0.3-3.0 |
| Manganese (II) | 0.4-2.5 |
| Copper (II) | 0.05-0.25 |
| Zinc (II) | 0.3-2.5 |
| Cobalt (II) | 0.005-0.05 |
| Selenium (IV) | 0.01-0.03 |
| Iodine (I) | 0.03-0.08 |

The rest is water.

2. A microelement veterinary preparation, according to claim 1, which characteristic feature is that it contains chlorides, sulfates, carbonates or oxides of iron, magnesium, manganese, copper, zinc, and cobalt (in the form of hydrates or anhydrous salts) as their salts, it also includes sodium selenite or selenious acid as selenium salt, and potassium iodide, or sodium, or elemental iodine as iodine salt.
